(19) United States
(12) Patent Application Publication
(10) Pub. No.: US 2025/0022556 A1
(43) Pub. Date: Jan. 16, 2025

(54) Systems and Methods Enabling Baseline Prediction Correction

(71) Applicant: Unlearn.AI, Inc., San Francisco, CA (US)

(72) Inventors: Luca D'Alessio, Medford, MA (US); Rishabh Gupta, Somerville, MA (US); Charles Kenneth Fisher, Truckee, CA (US)

(73) Assignee: Unlearn.AI, Inc., San Francisco, CA (US)

(21) Appl. No.: 18/669,364

(22) Filed: May 20, 2024

Related U.S. Application Data
(63) Continuation of application No. 18/340,715, filed on Jun. 23, 2023, now Pat. No. 12,020,789.
(60) Provisional application No. 63/485,771, filed on Feb. 17, 2023.

(51) Int. Cl. G16H 10/60; G06N 3/02

(52) U.S. Cl. CPC G16H 10/60; G06N 3/02

(57) ABSTRACT

Systems and techniques for time-series forecasting are illustrated. One embodiment includes a method for refining time-series forecasts, the method obtains timestep information including baseline information, a time gap, and context information. The baseline information includes information known about the system at a time when the multivariate time-series is generated. The context information includes at least one vector of time-independent background variables related to the system. The method determines, based on the timestep information, parameter predictions for the system at a first timestep and a second timestep. The method derives actual state values for the system at the first timestep. The method updates the parameter predictions for the system at the second timestep, using a gating function, based on a discrepancy between: the parameter predictions for the system at the first timestep, and the actual state values for the system at the first timestep.

### CROSS-REFERENCE TO RELATED APPLICATIONS

The current application is a continuation of U.S. patent application Ser. No. 18/340,715 entitled “Systems and Methods Enabling Baseline Prediction Correction” filed Jun. 23, 2023, which claims the benefit of and priority under 35 U.S.C. § 119 (e) to U.S. Provisional Patent Application No. 63/485,771 entitled “Baseline Predictor Corrector” filed Feb. 17, 2023, the disclosures of which are hereby incorporated by reference in its entirety for all purposes.

### FIELD OF THE INVENTION

The present invention generally relates to data analysis and application and, more specifically, time-series forecasting.

### BACKGROUND

Time-series forecasting refers to computational trend analysis methods that are frequently used to make informed decisions, respond to environmental changes, and determine responses for industries such as healthcare and research. Time series forecasting generally involves making long-term predictions based on historical analysis and assessments of change over time. Time-series forecasting methods include recursive forecasting and direct multi-step forecasting, both of which carry their own benefits. Recursive forecasting is a method where predictions for future time steps are made one step at a time, using information (including other, earlier, forecasts) from previous time steps. In such cases, a single time series model is estimated, while the input for the next timestep is the output for the previous timestep, thereby functioning in a manner both conceptually simple and data efficient. Direct multi-step forecasting, on the other hand, involves using all observed/historical information to make predictions for multiple future time steps in one go. By training a model in full for multiple time steps, the approach avoids the accumulation of errors and leads to predictions with lower bias.

### SUMMARY OF THE INVENTION

Systems and techniques for time-series forecasting are illustrated. One embodiment includes a method for refining time-series forecasts, the method obtains timestep information concerning a multivariate time-series generated for a system. The timestep information includes baseline information, a time gap, and context information. The baseline information includes information known about the system at a time when the multivariate time-series is generated. The context information includes at least one vector of time-independent background variables related to the system. The method determines, based on the timestep information, parameter predictions for the system at a first timestep and a second timestep, wherein the second timestep occurs after the first timestep and a time difference between the first timestep and the second timestep corresponds to the time gap. The method derives actual state values for the system at the first timestep. The method updates the parameter predictions for the system at the second timestep, using a gating function, based on a discrepancy between: the parameter predictions for the system at the first timestep, and the actual state values for the system at the first timestep.

In a further embodiment, the parameter predictions are selected from the group consisting of: predictions for a state of the system; and predictions for a variance of the system.

In a still further embodiment, when the parameter predictions are predictions for the state of the system, updating the parameter predictions is performed using the formula: y_{t+dt}+(xₜ−yₜ)e^{-λdt}. In the formula, A corresponds to an array of learnable parameters; dt corresponds to the time gap; xₜ corresponds to the actual state values for the system at the first timestep; yₜ corresponds to predictions for the state of the system at the first timestep; and y_{t+dt} corresponds to predictions for the state of the system at the second timestep.

In another embodiment, when the parameter predictions are predictions for the variance of the system, updating the parameter predictions is performed using the formula: (1−e^{-λ2dt})s_{t+dt}². In the formula, λ corresponds to an array of learnable parameters; dt corresponds to the time gap; and s_{t+dt}² corresponds to predictions for the variance of the system at the second timestep.

In yet another embodiment, updating the predictions for the variance of the system at the second timestep involves application of a softplus activation function.

In still yet another embodiment, the system corresponds to an experimental assessment, the multivariate time-series corresponds to a set of states of participants to the experimental assessment, and the baseline information includes data to be monitored in the experimental assessment.

In a further embodiment, each of the actual state values for the system correspond to observations for one or more participants to the experimental assessment, and each of the parameter predictions for the system correspond to predictions for one or more participants to the experimental assessment.

In another embodiment, the system corresponds to an assessment of a condition of a patient.

In still another embodiment, the multivariate time-series corresponds to a health history of the patient.

In a further embodiment, each of the actual state values for the system correspond to observations for one or more participants to the experimental assessment.

In another further embodiment, each of the parameter predictions for the system correspond to predictions for one or more participants to the experimental assessment.

In yet another embodiment, the baseline information includes information used in a recent diagnosis of the patient.

In still yet another embodiment, at least one of the parameter predictions for the system is applied to a medical diagnosis of the patient.

In another embodiment, the system corresponds to an assessment of a condition of a patient.

In a further embodiment, the multivariate time-series corresponds to a health history of the patient.

In another further embodiment, the baseline information includes information used in a recent diagnosis of the patient.

In yet another further embodiment, at least one of the parameter predictions for the system is applied to a medical diagnosis of the patient.

In another embodiment, parameter predictions for the system are determined by a neural network with learnable parameters.

In a further embodiment, the neural network is at least one of the group consisting of: a multilayer perceptron (MLP); and a residual neural network.

In a still further embodiment, the MLP includes a single linear layer.

In another embodiment the neural network takes, as a particular input, at least one from the group consisting of: a product of variables of the baseline information and time, and a product of variable of the context information and time.

In a further embodiment, the neural network automatically learns the particular input.

In another embodiment, the baseline information reflects a state of the system at a pre-determined point in the multivariate time-series.

In another embodiment, updating the parameter predictions for the system at the second timestep is further based on the time gap.

In a further embodiment, a determined length of the time gap is inversely proportional to weight given to the discrepancy in updating the parameter predictions for the system at the second timestep.

In another embodiment, the parameter predictions are anchored to the baseline information.

In still another embodiment, updating the parameter predictions for the system at the second timestep includes producing an adjustment term, wherein the adjustment term includes the gating function and the discrepancy, and

In a further embodiment, updating the parameter predictions for the system at the second timestep includes adding the adjustment term to the parameter predictions for the system at the second timestep.

In another further embodiment, producing the adjustment term includes multiplying the gating function by the discrepancy.

In another embodiment, the method, when the discrepancy suggests that the parameter predictions for the first timestep is an underestimation, updates the parameter predictions for the second timestep includes increasing the parameter predictions for the second timestep.

In a further embodiment, the method, when the discrepancy suggests that the parameter predictions for the first timestep is an overestimation, updates the parameter predictions for the second timestep includes decreasing the parameter predictions for the second timestep.

In another embodiment, updating the parameter predictions for the system is performed on a recursive basis.

In yet another embodiment, the time-independent background variables include at least one selected from the group consisting of race, sex, disability, and genetic profile.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 conceptually illustrates a process for deriving and updating a time-series forecast generated in accordance with many embodiments of the invention.

FIG. 2 illustrates a system that provides for the gathering and distribution of data for producing time-series forecasts in accordance with numerous embodiments of the invention.

FIG. 3 illustrates a data processing element constructed in accordance with various embodiments of the invention.

FIG. 4 illustrates a data processing application configured in accordance with certain embodiments of the invention.

### DETAILED DESCRIPTION

Turning now to the drawings, systems and methods configured in accordance with various embodiments of the invention may enable precise, low-latency forecasting for fields including but not limited to healthcare diagnosis, treatment, and/or research. Systems configured in accordance with some embodiments of the invention may be referred to as Baseline Predictor-Correctors in this application. Many problems (e.g., modeling patient trajectories) require the ability to generate time-series. That is, to generate a sequence of states {v(t)}ₜ₌₀^{τ}. Baseline Predictor-Correctors configured in accordance with many embodiments can incorporate predictor models (f) to provide best guess predictions at two or more time-series time points (t, t+dt) based on information including but not limited to previous timestep state estimates, observed information, and baseline information (x₀) (i.e., information known before any forecasting is performed). Additionally or alternatively, best guess predictions may depend on static context information including but not limited to time-independent background variables. Once best guess predictions are obtained systems may utilize corrector models (h) to adjust the predictor based on variables including but not limited to the change in time (dt) and values derived from discrepancies between the estimated change(s) and the observed change(s).

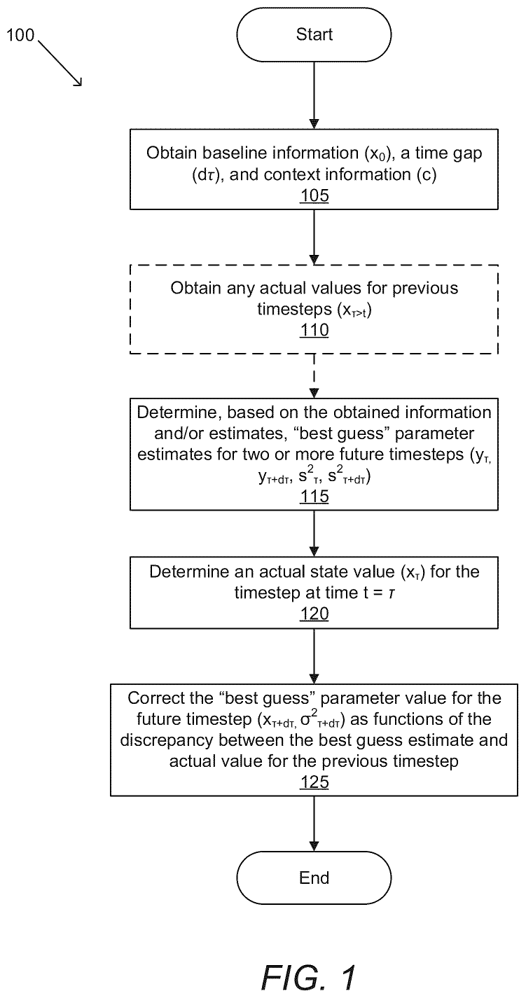

A process for deriving and updating a time-series forecast generated in accordance with many embodiments of the invention, is illustrated in FIG. 1. Process 100 obtains (105) baseline information (x₀), a time gap (dτ), and context information (c). In accordance with certain embodiments of the invention, context information may take the form of vectors of time-independent background variables. Within healthcare and/or clinical trials, context information may include but is not limited to pre-treatment covariates such as race, sex, disability, and/or genetic profile. Additionally or alternatively baseline information may incorporate various types of information known before and/or at the start of forecasting attempts. In particular, such characteristics may be used to answer, through time-series configured in accordance with some embodiments, inquiries such as “Given a subject's baseline characteristics, how will those characteristics evolve in time?” As such, in accordance with some embodiments, baseline information for individual clinical trials may include but is not limited to T-cell count, bone density, and/or BMI.

Process 100 may also obtain (110) any actual state values for previous timesteps (x_{τ>t}), wherein the actual state values reflect the state of the system being forecast. In accordance with many embodiments of the invention, (x_{τ}; τ>0) may disclose soon-to-be observed states, that correspond to time t=τ>0. As such, the time gap (dτ) may refer to the difference in time between time t=τ and the time that is to be observed further in the future, (τ+dτ; τ>0; dτ>0). In accordance with some embodiments, observed states may correspond to one or more trial participants.

Process 100 determines (115), based on the obtained information and/or actual values, “best guess” parameter estimates for two or more future timesteps. In accordance with numerous embodiments of the invention, parameters may include but are not limited to state estimates (also referred to as “expected values” in this disclosure) and/or variance estimates (x_{τ}, x_{τ+dτ}, σ_{τ}², σ_{τ+dτ}²). Best guess parameters may be produced by predictor models (“predictors”). In accordance with multiple embodiments, predictors (e.g., f₁, f₂) may provide best guess parameters including but not limited to state and/or variance estimates for time t=τ and/or time t=τ+dτ conditioned on the baseline (x₀) and context (c) information and/or the various previous timestep information available. This may enable best guess parameters including but not limited to best guesses for expected values, i.e., state estimates (yₜ):

$$\begin{matrix}{y}_{τ} := {f}_{1} ( {x}_{0} , c , τ ) \approx E [ {x}_{τ} \vert {x}_{0} , c , τ ] \\ {y}_{τ + d τ} := {f}_{1} ( {x}_{0} , c , τ + d τ ) \approx E [ {x}_{τ + d τ} \vert {x}_{0} , c , τ + d τ ]\end{matrix} \qquad ( 1 A )$$

and best guesses for variance (sₜ²):

$$\begin{matrix}{s}_{τ}^{2} := {f}_{2} ( {x}_{0} , c , τ ) \approx Var [ {x}_{τ} \vert {x}_{0} , c , τ ] \\ {s}_{τ + d τ}^{2} := {f}_{2} ( {x}_{0} , c , τ + d τ ) \approx Var [ {x}_{τ + d τ} \vert {x}_{0} , c , τ + d τ ]\end{matrix} \qquad ( 1 B )$$

Process 100 determines (120) an actual state value (e.g., x_{τ}) for the timestep at time t=τ. In accordance with various embodiments of the invention, once the actual state value for time t=τ is obtained (120), comparisons to the best guess state estimates (y_{τ}) for time t=τ may be generated. Additionally or alternatively, comparisons to other best guess parameters, including but not limited to variance (s_{τ}²) for time t=τ may be generated.

Process 100 corrects (125) the “best guess” parameter estimates for the future timestep (e.g., y_{τ+dτ}; s_{τ+dτ}²) as functions of the discrepancy between the state estimate and the actual state value for the previous timestep. As indicated above, these corrections may be performed on, but are not limited to state estimates (y_{τ+dτ}) and/or variance estimates (s_{τ+dτ}²) Corrector models (“correctors”) may be used to correct the best guess at time τ+dτ using the actual information from the previous time step x_{τ}. In accordance with various embodiments, when the prediction of the predictor f is undershooting the actual state values at time τ, correctors (h₁, h₂) may conclude a high likelihood that f will undershoot state estimate y_{τ+dτ}. In such instances, h may determine that the output should be a positive contribution. Additionally or alternatively, when predictor f overshoots at time τ then h may conclude a high likelihood that f will overshoot state estimates (e.g., x_{τ+dτ} will overshoot y_{τ+dτ}; s_{τ+dτ}² will overshoot σ_{τ+dτ}²)

The final prediction may then take the form:

$$E [ {x}_{τ + d τ} \vert {x}_{0} , {x}_{τ} , τ , d τ , c ] \approx {h}_{1} ( {y}_{τ} , {y}_{τ + d τ} , {s}_{τ}^{2} , {s}_{τ + d τ}^{2} , {x}_{τ} , d τ ) \qquad ( 2 A )$$

$$Var [ {x}_{τ + d τ} \vert {x}_{0} , {x}_{τ} , τ , d τ , c ] \approx {h}_{2} ( {y}_{τ} , {y}_{τ + d τ} , {s}_{τ}^{2} , {s}_{τ + d τ}^{2} , {x}_{τ} , d τ ) \qquad ( 2 B )$$

wherein h_{(·)}(y_{τ}, y_{τ+dτ}, s_{τ}², s_{τ+dτ}², x_{τ}, dτ) represents the corrector model configuration. As such, the correctors may update estimates based on inputs including but not limited to initial best guess parameter estimates for the two or more timesteps τ (y_{τ}, s_{τ}²) and τ+dτ (y_{τ+dτ}, s_{τ+dτ}²), actual state values for timestep τ (x_{τ}), and/or time lags between the timesteps (dτ). In accordance with various embodiments of the invention, the outputs of the correctors may thereby be consistent.

In accordance with many embodiments of the invention, correctors may, additionally or alternatively, depend on time gap(s) dτ. When time gaps are very large, correctors may determine that discrepancies at time τ have less weight in regards to how the correction should be applied at τ+dτ. In such cases, systems configured in accordance with numerous embodiments of the invention may, as dτ passes above a certain threshold, determine that predictions for parameters at timestep at τ+dτ can be effectively reduced to the initial best estimates. When this happened:

$$E [ {x}_{τ + d τ} \vert {x}_{0} , {x}_{τ} , τ , d τ , c ] \approx {y}_{τ + d τ} \qquad ( 3 A )$$

$$Var [ {x}_{τ + d τ} \vert {x}_{0} , {x}_{τ} , τ , d τ , c ] \approx {s}_{τ + d τ}^{2} \qquad ( 3 B )$$

Additionally or alternatively, when dτ is small, correctors may determine that discrepancies at time τ have more weight in regards to how the correction which should be applied at τ+dτ. In such cases, systems configured in accordance with many embodiments of the invention may, as dr passes under a particular threshold, determine that predictions for parameters at timestep at τ+dτ can be adjusted. In particular, in such cases, the resulting formulae may take the form:

$$\begin{matrix}E [ {x}_{τ + d τ} \vert {x}_{0} , {x}_{τ} , τ , d τ , c ] \approx {x}_{τ} \\ Var [ {x}_{τ + d τ} \vert {x}_{0} , {x}_{τ} , τ , d τ , c ] < min ( {s}_{τ + d τ}^{2} , {s}_{τ}^{2} )\end{matrix} \qquad ( 4 A )$$

Systems and methods configured in accordance with a number of embodiments of the invention can be applied recursively to generate long-term forecasts. At each recursion step, the predictions may be anchored to the baseline information thus preventing the accumulation of errors. By using the information available at the previous timestep, systems may generate long forecasts with precise time-correlation between timesteps.

In accordance with certain embodiments of the invention, for the first recursion step, x_{τ} may be unknown. In response to this, systems configured in accordance with many embodiments may follow Equations (1A) and (1B). Additionally or alternatively, systems configured in accordance with certain embodiments may set τ=0 such that x_{τ}=y_{τ}=x₀.

In accordance with some embodiments of the invention, predictive inferences made by predictors may take various forms. Predictors may operate as deterministic functions of their respective input values. In accordance with numerous embodiments, predictors can be implemented as Neural Networks with learnable parameters. Additionally or alternatively, predictors can be implemented as Multilayer perceptrons (MLPs). For example, certain predictors for state estimators (f₁) may follow the equation:

$${y}_{t} = {f}_{1} ( {x}_{0} , c , t ) = MLP ( {x}_{0} , c , t \times {x}_{0} , t \times c , t ) \qquad ( 5 A )$$

In accordance with certain embodiments, MLP predictors may receive, as input features, the product of baseline and context variables with time (t×x₀, t×c) in addition to the original variables.

In accordance with many embodiments of the invention, various types of feature engineering, including the above may be implemented by MLPs. For example, in accordance with numerous embodiments, predictors can be implemented as residual neural networks. For example, predictors for state estimates may follow the equation:

$${y}_{t} = {f}_{1} ( {x}_{0} , c , t ) = {x}_{0} + MLP ( t \times {x}_{0} , t \times c , t ) \qquad ( 5 B )$$

In accordance with many embodiments, predictors for variance (f₂) may include non-linearities including but not limited to softplus activation functions in order to enforce non-negative outputs. As such, predictors for variance estimates may follow the equation:

$${s}_{t}^{2} = {f}_{2} ( {x}_{0} , c , t ) = activation ( MLP ( {x}_{0} , c , t \times {x}_{0} , t \times c , t ) ) \qquad ( 6 A )$$

Additionally or alternatively, predictors for variance may follow the equation:

$${s}_{t}^{2} = {f}_{2} ( {x}_{0} , c , t ) = activation ( a ( {x}_{0} , c ) + b ( {x}_{0} , c ) \times t ) \qquad ( 6 B )$$

where a and b are non-negative learnable functions. In accordance with numerous embodiments, non-negative learnable functions can be implemented as, but are not limited to, MLPs. Additionally or alternatively, activation functions may be used here to control and/or cap variance growth over time.

In accordance with some embodiments of the invention, MLP predictors may be configured to automatically learn derived features including but not limited to the product(s) of baseline and context variables with time. Additionally or alternatively, systems may directly provide such variables to enable the use of shallower networks, improving training and which results in faster training and less over-fitting. In accordance with a few embodiments, MLPs can be reduced to single linear layers.

In accordance with many embodiments of the invention, corrections made by correctors may take numerous forms. In particular, corrector functions may need to be considered in tandem, due to prospective consistency requirements described above. In accordance with some embodiments, correctors can be implemented as gating functions. Additionally or alternatively, the gating function may be multiplied by discrepancies between actual state values and best guesses (e.g. (xₜ−yₜ)).

In accordance with certain embodiments, correctors for state estimates may utilize various gating functions that input variables including but not limited to state estimates (e.g., yₜ, y_{t+dt}), variance best guesses (e.g., sₜ², s_{t+dt}²), actual state values (e.g., xₜ) and/or time gaps (e.g., dt). For example, so correctors for state estimates may take the form:

$${h}_{1} ( {y}_{t} . {y}_{t + dt} , {s}_{t}^{2} , {s}_{t + dt}^{2} , {x}_{t} , dt ) = {y}_{t + dt} + ( {x}_{t} - {y}_{t} ) {e}^{- λ dt} \qquad ( 7 )$$

wherein λ represents an array of learnable parameters. In accordance with various embodiments of the invention, gating functions (e.g., e^{-λdt}) may depend on time gaps to reflect system expectations that, as dt increases, correction inferences will decrease to zero. Gating functions for state estimates configured in accordance with certain embodiments may interpolate between short time lag situations (e.g., where dt approaches 0 and h₁≈xₜ) and long time lag situations (e.g., where dt approaches ∞ and h₁≈y_{t+dt}). As such, discrepancies (at timestep t) between predictions and actual values may be modified accordingly.

In accordance with numerous embodiments, the corrector for state estimates may pose constraints on the form of correctors for other parameters, including but not limited to variance. Gating functions for variance configured in accordance with multiple embodiments may similarly interpolate between short time lag situations (e.g., where dt approaches 0 and h₂≈0) and long time lag situations (e.g., where dt approaches ∞ and h₂≈s_{t+dt}²). For example, one such version of the gating function for variance correctors may be:

$${h}_{2} ( {y}_{t} . {y}_{t + dt} , {s}_{t}^{2} , {s}_{t + dt}^{2} , {x}_{t} , dt ) = ( 1 - {e}^{- λ2 dt} ) {s}_{t + dt}^{2} \qquad ( 8 )$$

which can enable h₂ to remain non-negative.

While specific processes for generating time-series forecasts are described above, any of a variety of processes can be utilized to forecast time-series as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

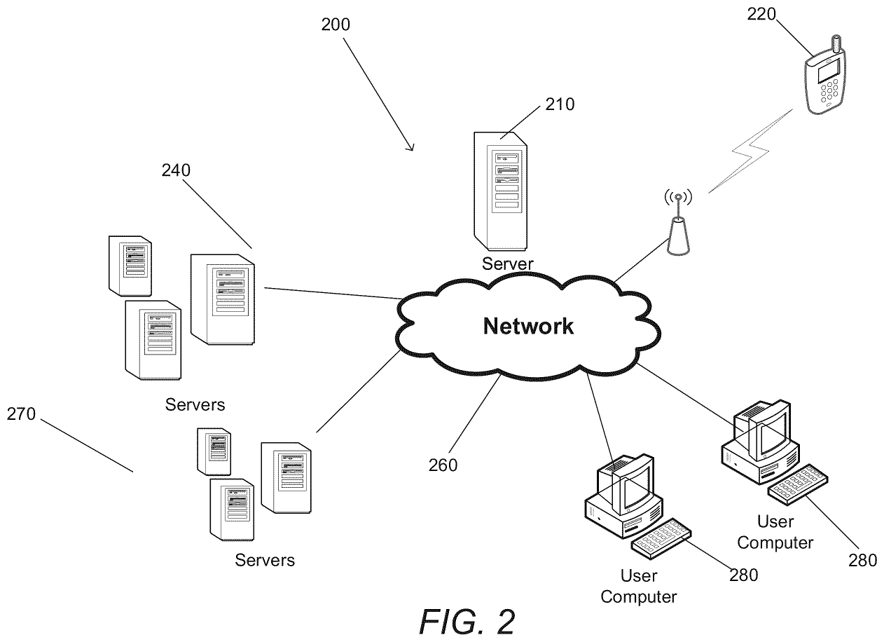

A system that provides for the gathering and distribution of data for forecasting time-series in accordance with some embodiments of the invention is shown in FIG. 2. Network 200 includes a communications network 260. The communications network 260 is a network such as the Internet that allows devices connected to the network 260 to communicate with other connected devices. Server systems 210, 240, and 270 are connected to the network 260. Each of the server systems 210, 240, and 270 is a group of one or more servers communicatively connected to one another via internal networks that execute processes that provide cloud services to users over the network 260. For purposes of this discussion, cloud services are one or more applications that are executed by one or more server systems to provide data and/or executable applications to devices over a network. The server systems 210, 240, and 270 are shown each having three servers in the internal network. However, the server systems 210, 240 and 270 may include any number of servers and any additional number of server systems may be connected to the network 260 to provide cloud services. In accordance with various embodiments of this invention, a network that uses systems and methods that forecast and/or modified time-series in accordance with an embodiment of the invention may be provided by a process (or a set of processes) being executed on a single server system and/or a group of server systems communicating over network 260.

Users may use personal devices 280 and 220 that connect to the network 260 to perform processes for providing and/or interaction with a network that uses systems and methods that m in accordance with various embodiments of the invention. In the shown embodiment, the personal devices 280 are shown as desktop computers that are connected via a conventional “wired” connection to the network 260. However, the personal device 280 may be a desktop computer, a laptop computer, a smart television, an entertainment gaming console, or any other device that connects to the network 260 via a “wired” connection. The mobile device 220 connects to network 260 using a wireless connection. A wireless connection is a connection that uses Radio Frequency (RF) signals, Infrared signals, or any other form of wireless signaling to connect to the network 260. In FIG. 2, the mobile device 220 is a mobile telephone. However, mobile device 220 may be a mobile phone, Personal Digital Assistant (PDA), a tablet, a smartphone, or any other type of device that connects to network 260 via wireless connection without departing from this invention.

As can readily be appreciated the specific computing systems used to manage time-series applications are largely dependent upon the requirements of a given application and should not be considered as limited to any specific computing system(s) implementation.

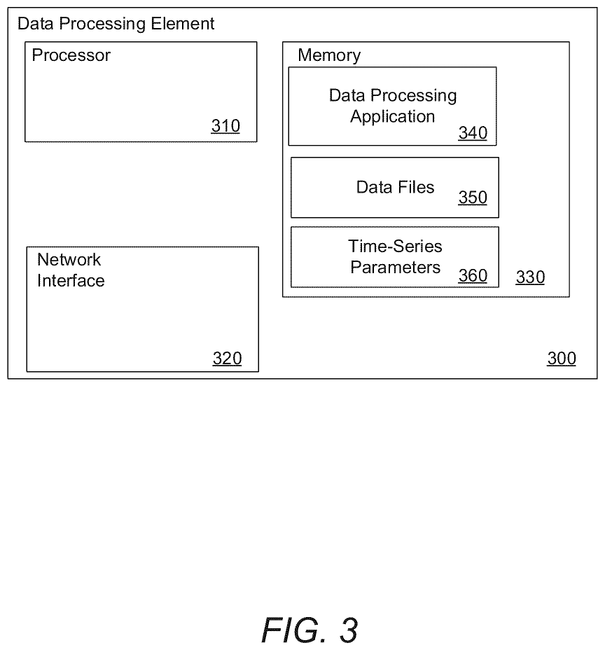

An example of a data processing element for training and utilizing a generative model in accordance with a number of embodiments is illustrated in FIG. 3. In various embodiments, data processing element 300 is one or more of a server system and/or personal devices within a networked system similar to the system described with reference to FIG. 2. Data processing element 300 includes a processor (or set of processors) 310, network interface 320, and memory 330. The network interface 320 is capable of sending and receiving data across a network over a network connection. In a number of embodiments, the network interface 320 is in communication with the memory 330. In several embodiments, memory 330 is any form of storage configured to store a variety of data, including, but not limited to, a data processing application 340, data files 350, and time-series parameters 360. Data processing application 340 in accordance with some embodiments of the invention directs the processor 310 to perform a variety of processes, including but not limited to the process disclosed in FIG. 1. Processor 310 may use data from data files 350 to update time-series parameters 360 in order to allow the processor 310 to determine, update, and/or apply time-series forecasts in accordance with many embodiments of the invention.

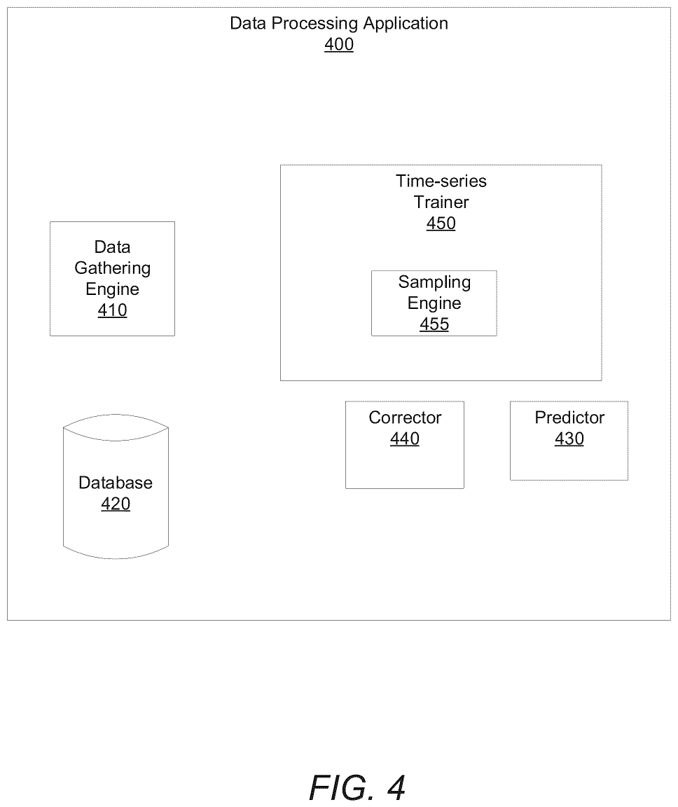

A data processing application in accordance with a number of embodiments of the invention is illustrated in FIG. 4. In accordance with many embodiments of the invention, data processing applications 400 may be used to produce and/or modify time-series forecasts. In this example, data processing application 400 includes a data gathering engine 410, database 420, a predictor 430, a corrector 440, and a time-series trainer 450. The trainers 450 may include but are not limited to sampling engine(s) 455 which can be used to perform random sampling processes used in training predictors 430 and/or correctors 440. Data processing applications in accordance with many embodiments of the invention process data to generate and/or modify forecasts.

Databases in accordance with various embodiments of the invention store data for use by data processing applications, including (but not limited to) input data, pre-processed data, time-series parameters, and output data. In some embodiments, databases may be located on separate machines (e.g., in cloud storage, server farms, networked databases, etc.) from data processing applications.

As described above, as a part of the data-gathering process, the data in accordance with several embodiments of the invention may be pre-processed in order to simplify the data. Unlike other pre-processing which is often highly manual and specific to the data, this can be performed automatically based on the type of data, without additional input from another person.

Although specific examples of data processing elements and applications are illustrated in FIGS. 3-4, any of a variety of data processing elements and/or applications can be utilized to perform processes for facilitating time-series generation and modification similar to those described herein as appropriate to the requirements of specific applications in accordance with embodiments of the invention.

Even though it may only be possible to predict the probability of a health outcome for an individual patient, time-series configured in accordance with many embodiments of the invention can make it possible to precisely predict the number of patients with that health outcome in a large population. For example, predicting health risks may make it possible to accurately estimate the cost of insuring a population. Similarly, predicting the likelihood that a patient will respond to a particular therapeutic may make it possible to estimate the probability of a positive outcome in a clinical trial.

Developing the ability to accurately predict patients' prognoses is a necessary step towards precision medicine. A patient can be represented as a collection of information that describes their symptoms, their genetic information, results from diagnostic tests, any medical treatments they are receiving, and other information that may be relevant for characterizing their health. A vector containing this information about a patient may be described as a phenotype vector. A method for prognostic prediction in accordance with many embodiments of the invention uses past and current health information about a patient to predict a health outcome at a future time.

A patient trajectory, in accordance with numerous embodiments of the invention, may refer to time-series that describe patients' detailed health status(es) (e.g., a patient's phenotype vector) at various points in time. In several embodiments, prognostic prediction may take in a patient's trajectory (i.e., their past and current health information) and output predictions about a specific future health outcome (e.g., the likelihood they will have a heart attack within the next 2 years). By contrast, predicting a patient's future trajectory may involve predicting all of the information that characterizes the state of their health at all future times.

To frame this mathematically, let v (t) be a phenotype vector containing all of the information characterizing the health of a patient at time t. Therefore, a patient trajectory is a set {v(t)}ₜ₌₀^{T}. In some embodiments of the invention, models for simulating patient trajectories use discrete time steps (e.g., one month). Many of the examples are described with discrete time steps (e.g., τ=one month), but one skilled in the art will recognize that this is not necessary and that various other time steps can be employed in accordance with various embodiments of the invention. The length of the time step(s) in accordance with a number of embodiments of the invention may be selected to approximately match the frequency of treatment.

Additionally or alternatively, clinical decision support systems may provide information to patients, physicians, and/or other caregivers to help guide choices about patient care. Simulated patient trajectories may be applied to provide insights into a patient's future health that can inform choices of care. For example, a physician or caregiver can benefit from knowing the risks that a patient with mild cognitive impairment progresses to Alzheimer's disease, and/or that he or she begins to exhibit other cognitive or psychological systems. In certain embodiments, systems based on simulated patient trajectories can forecast these risks to guide care choices. Aggregating such predictions over a population of patients can also help estimate population level risks, enabling long-term planning by organizations, such as elder care facilities, that act as caregivers to large groups of patients. In some cases, models that are trained on data with treatment information would contain variables that describe treatment choices. Such a model could be used to assess how different treatment choices would change the patient's future risks by comparing simulated outcome risks conditioned on different treatments. In many embodiments, a caretaker or physician can treat a patient based on the treatment choices and/or the simulated trajectories.

Although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

1. (canceled)

2. A method for refining time-series forecasts, the method comprising:
determining, based on timestep information, parameter predictions for a system at a first timestep and a, later, second timestep, wherein:
the timestep information:
concerns a multivariate time-series generated for the system; and
comprises at least one of:
baseline information comprising information known about the system at a time when the multivariate time-series is generated; or
context information comprising at least one vector of time-independent background variables related to the system; and
the parameter predictions for each timestep comprise respective predictions for a state of the system at that timestep; and
updating the parameter predictions for the system at the second timestep, using a gating function, wherein a degree of modification for the updating is based on a discrepancy between:
the parameter predictions for the system at the first timestep; and
actual state values for the system at the first timestep.

3. The method of claim 2, wherein:
the system corresponds to an assessment of a condition of a patient; and
at least one of the parameter predictions for the system is applied to a medical diagnosis of the patient and is anchored to the baseline information.

4. The method of claim 3, wherein:
the baseline information comprises information used in a recent diagnosis of the patient;
the time-independent background variables comprise at least one selected from the group consisting of race, sex, disability, and genetic profile; and
the multivariate time-series corresponds to a health history of the patient.

5. The method of claim 2, wherein parameter predictions for the system are determined by a neural network with learnable parameters that is at least one of the group consisting of:
a multilayer perceptron (MLP); and
a residual neural network.

6. The method of claim 5, wherein the MLP comprises a single linear layer.

7. The method of claim 2, wherein:
updating the parameter predictions for the system at the second timestep is further based on a time gap corresponding to a time difference between the first timestep and the second timestep; and
a determined length of the time gap is inversely proportional to weight given to the discrepancy in updating the parameter predictions for the system at the second timestep.

8. The method of claim 2, further comprising:
when the discrepancy suggests that the parameter predictions for the first timestep is an underestimation, updating the parameter predictions for the second timestep comprises increasing the parameter predictions for the second timestep; and
when the discrepancy suggests that the parameter predictions for the first timestep is an overestimation, updating the parameter predictions for the second timestep comprises decreasing the parameter predictions for the second timestep.

9. The method of claim 2, wherein:
updating the parameter predictions for the system at the second timestep comprises:
producing an adjustment term, wherein the adjustment term comprises the gating function and the discrepancy; and
adding the adjustment term to the parameter predictions for the system at the second timestep.

10. The method of claim 9, wherein producing the adjustment term comprises multiplying the gating function by the discrepancy.

11. The method of claim 2, wherein updating the parameter predictions for the system is performed on a recursive basis.

12. A non-transitory computer-readable medium comprising instructions that, when executed, are configured to cause a processor to perform a process for refining time-series forecasts, the process comprising:
determining, based on timestep information, parameter predictions for a system at a first timestep and a, later, second timestep, wherein:
the timestep information:
concerns a multivariate time-series generated for the system; and
comprises at least one of:
baseline information comprising information known about the system at a time when the multivariate time-series is generated; or
context information comprising at least one vector of time-independent background variables related to the system; and
the parameter predictions for each timestep comprise respective predictions for a state of the system at that timestep; and
updating the parameter predictions for the system at the second timestep, using a gating function, wherein a degree of modification for the updating is based on a discrepancy between:
the parameter predictions for the system at the first timestep; and
actual state values for the system at the first timestep.

13. The non-transitory computer-readable medium of claim 12, wherein:
the system corresponds to an assessment of a condition of a patient; and
at least one of the parameter predictions for the system is applied to a medical diagnosis of the patient and is anchored to the baseline information.

14. The non-transitory computer-readable medium of claim 13, wherein:
the baseline information comprises information used in a recent diagnosis of the patient;
the time-independent background variables comprise at least one selected from the group consisting of race, sex, disability, and genetic profile; and
the multivariate time-series corresponds to a health history of the patient.

15. The non-transitory computer-readable medium of claim 12, wherein parameter predictions for the system are determined by a neural network with learnable parameters that is at least one of the group consisting of:
a multilayer perceptron (MLP); and
a residual neural network.

16. The non-transitory computer-readable medium of claim 15, wherein the MLP comprises a single linear layer.

17. The non-transitory computer-readable medium of claim 12, wherein:
updating the parameter predictions for the system at the second timestep is further based on a time gap corresponding to a time difference between the first timestep and the second timestep; and
a determined length of the time gap is inversely proportional to weight given to the discrepancy in updating the parameter predictions for the system at the second timestep.

18. The non-transitory computer-readable medium of claim 12, further comprising:
when the discrepancy suggests that the parameter predictions for the first timestep is an underestimation, updating the parameter predictions for the second timestep comprises increasing the parameter predictions for the second timestep; and
when the discrepancy suggests that the parameter predictions for the first timestep is an overestimation, updating the parameter predictions for the second timestep comprises decreasing the parameter predictions for the second timestep.

19. The non-transitory computer-readable medium of claim 12, wherein:
updating the parameter predictions for the system at the second timestep comprises:
producing an adjustment term, wherein the adjustment term comprises the gating function and the discrepancy; and
adding the adjustment term to the parameter predictions for the system at the second timestep.

20. The non-transitory computer-readable medium of claim 19, wherein producing the adjustment term comprises multiplying the gating function by the discrepancy.

21. The non-transitory computer-readable medium of claim 12, wherein updating the parameter predictions for the system is performed on a recursive basis.
